(19) United States
(12) Patent Application Publication
(10) Pub. No.: US 2016/0074297 A1
(43) Pub. Date: Mar. 17, 2016

(54) COMBINATION OF PETROSELINIC ACID AND TAURINE FOR ORAL ADMINISTRATION FOR HAIR AGING CONTROL

(71) Applicant: NUTRICOS TECHNOLOGIES, Clichy (FR)

(72) Inventors: Yann MAHE, Sainte Genevieve Des Bois (FR); Carole BRU, Courbevoie (FR); Isabelle CASTIEL, Nice (FR); Audrey GUENICHE, Rueil Malmaison (FR)

(21) Appl. No.: 14/890,069

(22) PCT Filed: May 6, 2014
(86) PCT No.: PCT/IB2014/061246

§ 371 Date: Nov. 9, 2015

(30) Foreign Application Priority Data
May 7, 2013 (FR) 13 54193

(51) Int. Cl. A61K 8/46; A61K 8/58; A61K 8/67; A23L 1/30; A23L 1/302; A23L 1/303; A23L 1/305; A23L 1/304; A61K 8/36; A61Q 5/00

(52) U.S. Cl. CPC A61K 8/466; A61K 8/361; A61K 8/58; A61K 8/67; A61K 8/678; A61Q 5/002; A23L 1/302; A23L 1/303; A23L 1/3051; A23L 1/304; A23L 1/30; A23V 2002/00

(57) ABSTRACT

The invention relates to the cosmetic use, by oral administration, of a combination of active agents including petroselinic acid and taurine, for improving hair quality and for preventing and controlling the microinflammation of hair follicles, in particular for controlling hair aging in men over the age of 30 or in women.

The present invention relates to the field of caring for keratin fibers, more particularly the hair. In particular, the invention is directed toward proposing a combination that is useful for improving the quality of the head of hair and for combating the aging of keratin fibers, and more particularly hair fibers, in particular causing thinning of the keratin fibers or more globally of the hair.

As will emerge hereinbelow, it is more particularly a matter in the context of the present invention of combating aging of the hair in the case of men over 30 years old or in the case of women.

Hairs are produced in hair follicles formed from epithelial sheaths of epidermal origin and from a hair bulb containing bulb keratinocytes in a constant state of division during the hair growth phases. Hair consists mainly of 85-90% protein.

The possession of healthy, strong hair throughout one's lifetime is an ambition of most women and men.

Thus, a first object of the invention is to propose a solution to men and women of any age for improving the quality of their head of hair.

In particular, men over 30 years old and women are subject to aging of their keratin fibers, in particular hair fibers. They may thus display alopecia related to the aging of the head of hair, which is characterized especially by global thinning and loss of hair matter and gradual lightening.

This is distinguished by certain aspects of alopecia known as androgenetic, the transmission of which is by definition hereditary, the progress of which is rapid and initially affects the forehead and the temples, and, on account of the role of the androgens, appears very early after puberty, mainly in young men.

Recent studies have demonstrated in the case of more elderly individuals, and without preference in the case of men and women, a form of alopecia that takes longer to become established, involving the contribution of a latent and silent form of inflammation (Pro-inflammatory cytokine cascade in human plucked hair Mahéet al. Skin Pharmacol. 1996, 366-375), known as low-noise inflammation and referred to as microinflammation (Mahéet al. Androgenetic alopecia and micro inflammation Int. J. Dermatol., 2000, 39, 576-584 and by Trueb, Clinical intervention in aging hair, 2006: 1(2) 121-129) as a contributory factor in the gradual involution of the hair follicles and miniaturization of the hair follicle.

This phenomenon known as microinflammation of the hair follicle was identified and described in Mahé et al. (International Journal of Dermatology, 2000, 39, 576-584) as being a slow, subtle and painless inflammation, which causes no redness, itching or heating, or any of the therapeutic disorders usually associated with inflammation of the scalp.

Although it may involve perifollicular lymphocytic or monocytic inflammatory cell infiltrates, it is markedly distinguished from “conventional” inflammation, which may take place in the dermis or the scalp, which manifests itself much more irritatingly, or even painfully, and which has much more destructive consequences on the scalp and on the hair fiber. In particular, it is distinguished from alopecia areata associated with an established immuno-inflammatory disorder which results in massive and occasionally localized and often spontaneously reversible loss of hair, affecting without preference men, women and children, which makes it possible in principle to exclude the contribution of androgens in its physiopathology.

Low-noise microinflammation contributes, on the contrary, toward more gradual and intrinsic aging of the scalp and of the hair. As indicated previously, the perifollicular and follicular microinflammation according to the invention may lead, besides thinning of the hair strands, to hair loss, and result in rather diffuse alopecia or even alopecia centered on the crown, in particular in the case of men more than 30 years old or in the case of women, as opposed to androgenetic alopecia which starts at the crown and the forehead and mainly affects young men.

Aging of the hair, characterized by this microinflammation, is in fact a natural phenomenon which takes place gradually and more or less early depending on the individual. This microinflammation is sparingly perceptible at the surface and especially has the consequence of triggering perifollicular dermal disturbances leading to thickening of the inner sheath of the hair and to its gradual miniaturization in the course of the successive cycles of regeneration of a new functional follicle. Thinning of the hair strand, a hair strand which is less thick and less resistant to traction and to breaking, and a decrease in the volume of the overall head of hair are thus observed. In addition, the scalp becomes visible in places, with pronounced balding of the crown, without, however, systematically greatly affecting the temples or the forehead. Occasionally even, this diffuse, late-onset and slowly-evolving alopecia may take over from or reinforce and accentuate the effects of an initial phase of androgenetic alopecia. In addition, aging of the hair may also be characterized by wear and impairment of the hair fiber, reflected by embrittled/sensitized hair, or alternatively dry hair.

Thus, it is clearly seen that this perifollicular microinflammation, which is representative of intrinsic aging of the hair bulb and the hair fibers, is only the cause of cosmetic disorders, and especially of those listed previously. In contrast with conventional inflammation, the associated disorders of which fall within the therapeutic field, the prevention and/or treatment of clinically silent and low-noise microinflammation of the hair follicle, and thus of the disorders associated therewith, fall only within the cosmetic field.

There is thus also a need for active agents, or even a combination of active agents, which are capable of preventing and/or combating microinflammation of the hair follicles.

Many cosmetic compositions containing amino acids, in particular glutamine, arginine and cysteine, or peptides and/or taurine, exist for treating the scalp.

By way of example, document WO 2004/000293 describes the use of taurine and/or hypotaurine and/or acceptable salts thereof for the preparation of an oral composition that is useful in the treatment and prevention of aging of the pilosebaceous unit and/or of alopecia.

In addition, document WO 2008/071897 describes a composition comprising petroselinic acid for topical application to the hair, for treating or preventing its impairment, and for improving the quality of keratin fibers.

However, consumers are always in search of active agents or of combinations of active agents that are more effective for improving the quality of their head of hair. In addition, no active agents that are specifically suited to preventing and combating low-noise microinflammation of the hair follicle and for combating gradual thinning of the hair strands, in particular in the case of men over 30 years old and in the case of women, exist at the present time.

The present invention proposes to satisfy this need by proposing a combination of petroselinic acid with taurine, administered orally.

As illustrated in the examples of the present application, the inventors have in point of fact noted that a combination of active agents in accordance with the invention proves to be capable of synergistically increasing the amount of lipoxin A4. Lipoxin A4 belongs to the resolvin family. This family of compounds naturally produced by the body acts in a manner complementary to conventional anti-inflammatory agents by raising the threshold for triggering a “dermatologically conventional” inflammatory response, and more particularly so as to raise the threshold of appearance of the signals of this conventional inflammation, namely redness, pain and heat.

The main subject of the invention is thus the oral cosmetic use of a combination of active agents comprising petroselinic acid and taurine, for improving the quality of the head of hair, in particular in the case of men over 30 years old or in the case of women.

Preferably, according to the invention, the oral cosmetic use of a combination of active agents comprising petroselinic acid and taurine is characterized in that the improvement in the quality of the head of hair comprises an improvement in the sheen of the head of hair and/or an improvement in the stylability of the head of hair, and/or in its strength and hold and/or a decrease in hair loss and/or an improvement in the growth of the hair fiber and/or an improvement in the volume of the head of hair and/or an improvement in the quality of the hair fiber.

Even more preferably, the improvement in the volume of the head of hair comprises an improvement in and/or maintenance of the diameter of the hair fiber, and/or an improvement in the density and/or limitation of the thinning of the hair fiber.

Preferably also, the improvement in the quality of the hair fiber comprises improving the tensile strength of the hair, and/or the resistance of styling and/or shaping of the hair, and/or preventing and/or combating limp and/or breakable and/or dull and/or split and/or embrittled and/or sensitized and/or dry hair, and/or improving the softness and/or vigor of the hair fibers.

The present invention is also directed toward the oral cosmetic use of a combination of petroselinic acid and taurine for combating aging of the hair in the case of men over 30 years old or in the case of women.

The present invention is directed in particular toward the oral cosmetic use of a combination of petroselinic acid and taurine for preventing and/or combating low-noise microinflammation of the hair follicles and/or of the scalp, in particular of the hair follicles.

According to the invention, the term “aging of the hair” is intended to denote a change in the appearance of the hair fiber (thin, dull hair with no strength (limp) and with no radiance), which has a tendency to drop out, which becomes renewed less quickly, for which the collagen network which supports it is impaired by the substantial release of collagenase and the disruption of its network, for which the dermal and epithelial parts become rigidified, for which the synthesis of sebum becomes reduced, leading to dryness of the scalp and proportionately increasing the dull appearance and absence of radiance of the hair, for which the epidermis and the dermis of the scalp are also subjected to disruption of the collagen network, which rigidifies the dermis around the hair follicle, resulting in negative effects for the resident follicles and the implantation of new hairs.

The term “aging of the hair” also means a decrease in hair density, and also in the diameter of the hair strands, reflected by a pronounced or diffuse decrease in the coverage of the scalp.

According to the invention, the term “thinning of the hair strands” is intended to denote a decrease in the diameter of the hair strands below 40μm. The diameter of the hair strands may be advantageously measured using a Trichoscan machine, which is an automated equivalent of the Trichogram, in which the human eye has been replaced with image analysis software (Gasmueller, 2009). Moreover, below 40 m, the hair strands are difficult to see with the naked eye. The thinning of the hair strands may thus be perceived. A threshold of 5% may be considered as significant.

According to the invention, the term “growth of thick hair strands” is intended to denote the growth of hair strands with a diameter of greater than 40 μm, which may be measured by means of the Trichoscan machine, but which are also readily visible to the naked eye.

According to the invention, the term “increasing the hair density” means a larger number of hairs per cm². The hair density may also be measured with a Trichoscan machine. With this apparatus, a density of less than 280 hairs per cm² is considered to be a low density. A 5% increase in density may be considered as clinically significant and visible.

According to the invention, the term “preventing hair loss” means preventing a decrease in the percentage of hair strands in the telogenic phase, which may be measured with a Trichoscan machine, based on the fact that the hair strands in the telogenic phase no longer grow, in contrast with the hair strands in the anagenic phase. A 5% decrease in the number of hair strands in the telogenic phase may be considered as significant.

According to the invention, the term “preventing hair loss” also means preventing a decrease in the percentage of hair strands in the catagenic phase, which may be measured with a Trichogram, or by collecting hair strands on styling or after showering, based on the fact that the hair strands in the catagenic phase are the visible reflection of the hair strands previously engaged in the phase of growth stoppage (telogenic phase) and represent the loss as really perceived by an individual suffering from abnormally high hair loss. A 5% decrease in the number of hair strands in the catagenic phase may be considered as significant.

According to the invention, the term “increasing the overall volume of the head of hair” is intended to denote an increase in the diameter of the hairs, associated with a decrease in the heterogeneity of the diameter of the hair strands and with an increase in the number of hairs per cm².

According to the invention, the term “preventing and/or combating limp and/or breakable and/or dry and/or embrittled and/or dull and/or split hair” is intended to denote an overall improvement in the structure of the hair shaft and in particular of the cuticle, the outermost layer of the hair strand.

According to the invention, the term “improving the softness of the hair” is intended to denote an improvement in the condition of the scales of the cuticle and more particularly in their cohesion. Disjointed scales give the hair a coarse feel.

According to the invention, the term “improving the tensile strength of the hair and the vigor of the hair fibers” is intended to denote an improvement in the solidity of the hair, which may be measured by the tensile test. This measurement for determining the tensile mechanical properties of the hair is performed using a commercial tool, the MTT600 (mini Tensile Tester) from the company Dia Stron. A 5% increase in the force required to break the hair is considered as significant.

According to the invention, the term “stylability of the head of hair” is intended to denote a hair that is easy to comb and/or to brush. Disjointed or damaged scales become hooked into the scales of neighboring hair strands, causing entanglement of the head of hair, which then promotes the appearance of knots, making styling more difficult.

The term “stylability” also means better hold of the hairstyle after inserting curlers, blow-drying, relaxing or curling the hair.

According to the present invention, the term “sheen of the head of hair” is intended to denote reduced wear and/or repair of the scales of the cuticle to homogenize the surface of the hair and to promote the reflection of light.

Preferentially, the various parameters as defined above may be evaluated by men and/or women by means of a multiple-choice questionnaire in the context of an observational study that may be performed by dermatologists. An effect will be considered as significant if at least 50% of men and/or women have perceived a positive effect.

Men more than 30 years old and women are particularly concerned by the uses and processes according to the invention.

For the purposes of the present invention, the term “men” means the human male population.

A subject of the invention is also the use of a combination of active agents in accordance with the invention in the form of a food supplement.

A subject of the invention is also a cosmetic composition for oral administration or a food supplement containing a combination of petroselinic acid and taurine, in which the petroselinic acid is present in a content of between 10% and 70% by weight relative to the total weight of said combination of active agents.

A subject of the invention is also a cosmetic composition for oral administration or a food supplement containing a combination of petroselinic acid and taurine, zinc, or a salt thereof, preferably complexed with one or more (poly)hydroxy acids, preferably zinc gluconate, in which the petroselinic acid is present in a content of between 20% and 70% by weight relative to the total weight of said combination of active agents.

Preferably, such a composition or such a food supplement also comprises vitamin D3 and tocopheryl acetate.

According to the present invention, the term “taurine” means taurine, hypotaurine, or a salt thereof. Insofar as the combination according to the invention is intended for oral use in an individual, the salts that may be used are obviously chosen for their total harmlessness. Alkali metal or alkaline-earth metal salts, in particular magnesium salts, manganese, iron(II) or zinc(II) salts are suitable in this respect.

The content of taurine, hypotaurine or a salt thereof in a cosmetic composition intended for oral administration or in a food supplement in accordance with the invention may be between 1% and 40% of the total weight of the composition, especially between 5% and 40% of the total weight of the composition or of the supplement, particularly between 5% and 30% of the total weight of the composition or of the supplement.

The content of taurine, hypotaurine or a salt thereof in a cosmetic composition intended for oral administration or in an oral food supplement in accordance with the invention may be such that the daily dose of said taurine, hypotaurine or a salt thereof is between 4 and 700 mg/day and especially between 40 and 300 mg/day.

The invention also relates to a cosmetic process for
improving the quality of the head of hair; and/or
combating ageing of the hair in the case of men over 30 years old or in the case of women; and/or
preventing and/or combating low-noise microinflammation of the hair follicles and/or of the scalp;

comprising at least the oral administration, to an individual, of a combination of active agents in accordance with the invention, or of an oral composition or of a food supplement comprising such a combination of active agents.

The processes according to the invention have the characteristics of cosmetic processes especially insofar as they make it possible to improve the esthetics of the head of hair, in particular by combating the progressive thinning of the hair strands which appears especially with age. In addition, a combination of active agents, a composition or a food supplement according to the invention may be used daily for several months, without a medical prescription. The present invention thus clearly lies outside the therapeutic field.

In particular, the cosmetic processes and/or uses of the invention make it possible to protect aged hair, especially in the case of men over 30 years old or in the case of women.

The invention also relates to a food supplement comprising one part of the compounds forming the combination of active agents in accordance with the invention in a first composition, and at least the other part of the compounds forming the combination of active agents in a second composition, as a kit or combination product for simultaneous use, separate use or sequential use over time.

It is understood in the context of the present invention that “the oral cosmetic use” covers the use of products administered orally, these products being, for example, in the form of a food supplement as outlined below. These products produce an esthetic and comfort effect on the head of hair, or alternatively an effect which has a beauty purpose, for example with a view to protecting it, keeping it in good condition, and especially making it more attractive, especially by increasing the volume and hold of the overall head of hair.

The term “petroselinic acid-rich oil” means an oil comprising at least 20% of petroselinic acid and more preferentially more than 30% of petroselinic acid.

Alternatively, petroselinic acid, or monounsaturated fatty acid (C18:1 n-12 or cis delta 6) or C18 delta-6-cis-octadecenoic acid, is used in a combination of active agents in accordance with the invention.

Umbellifera plants are plants whose flowers are arranged in umbels. Species that are particularly rich in petroselinic acid are Umbellifarea-Apiacea and Araliaceae. Plants of the Thapsia genus are also sources of petroselinic acid (Avato et al., Lipids, 2001, 36, 845). The species preferably used in the invention are coriander, chervil, carrot, celery, cumin, caraway, parsley and dill. The umbellifera plant oil used according to the invention may be extracted from the seeds of these umbellifera plants, for example by grinding or pressing, followed by refining. The umbellifera plant oil has a petroselinic acid content which varies according to the umbellifera plant seed from which it is extracted.

For the same umbellifera plant, the petroselinic acid content also varies according to the country of origin of the umbellifera plant and according to the extraction, which may be more or less complete.

Petroselinic acid is also an abundant compound (approximately 48%) of

Geranium sanguineum seed oil, and also of Coriandrum sativum coriander seed oil.

Thus, according to one embodiment, the use that is the subject of the present invention is such that petroselinic acid is used in the form of umbellifera plant oil or Geranium sanguineum oil, preferably in the form of coriander (Coriandrum sativum), chervil, carrot, celery, cumin, caraway, parsley or dill oil, preferentially in the form of an oil of coriander (Coriandrum sativum) seeds.

The petroselinic acid contents are variable depending on whether the combination of active agents in accordance with the invention is used in a cosmetic composition intended for oral administration or in a food supplement.

Contents are indicated below as a guide.

The petroselinic acid content in a cosmetic composition intended for oral administration or in a food supplement in accordance with the invention may be between 1% and 70% of the total weight, especially between 10% and 70% of the total weight and particularly between 15% and 70% of the total weight of the composition or of the supplement. The petroselinic acid content in a cosmetic composition intended for oral administration or in an oral food supplement in accordance with the invention may be such that the daily dose of said petroselinic acid is between 5 and 1000 mg/day and especially between 50 and 650 mg/day.

According to a preferred variant, a cosmetic composition or a food supplement in accordance with the invention also comprises taurine.

According to a preferred variant, a cosmetic composition or a food supplement in accordance with the invention also comprises vitamin D3.

According to a preferred variant, a cosmetic composition or a food supplement in accordance with the invention also comprises taurine and vitamin D3.

According to a preferred variant, a cosmetic composition or a food supplement in accordance with the invention also comprises tocopheryl acetate.

According to a preferred variant, a cosmetic composition or a food supplement in accordance with the invention also comprises at least one amino acid chosen from amino acids that are and/or are not constituents of proteins, in particular arginine, cysteine and/or methionine.

According to one embodiment, a combination of active agents, a cosmetic composition for oral administration or a food supplement in accordance with the invention may also comprise zinc, or a salt thereof, preferably zinc gluconate.

The term “zinc” means zinc or a salt thereof (zinc acetate, chloride, citrate, lactate, gluconate, lactate, oxide, carbonate or sulfate), in particular Zn(II) salts, preferably complexed with one or more (poly)hydroxy acids such as gluconate.

The term “(poly)hydroxy acid” means any carboxylic acid which comprises a linear or branched, and saturated or unsaturated, preferably saturated and/or linear, hydrocarbon-based chain, comprising from 1 to 10 carbon atoms and from 1 to 9 hydroxyl groups, and comprising from 1 to 4 carboxylic groups —C(O)—OH, at least one of said —C(O)—OH groups of which is in the carboxylate form —C(O)—O— complexed with the Zn atom, preferably Zn(II).

More particularly, the zinc salt according to the invention is complexed with two carboxylate groups such as that of formula (I):

R—C(O)—O—Zn—O—C(O)—R′ (I)

in which R and R′, which may be identical or different, represent a (C1-C6) (poly)hydroxyalkyl group, and also solvates thereof, such as hydrates, and enantiomers thereof.

Preferably, the compound of formula (I) is zinc gluconate.

According to a particular embodiment of the invention, the zinc is not a zinc oxide, but a zinc salt. The term “Zn(II)” means a zinc atom in oxidation state Zn^{2+.}

Insofar as the product according to the invention is intended for oral use in an individual, the zinc salts that may be used are obviously chosen for their total harmlessness.

The content of zinc, in particular of zinc gluconate, in a cosmetic composition intended for oral administration or in a food supplement in accordance with the invention may be between 0.001% and 30% by weight, especially between 0.01% and 25% by weight and particularly between 0.1% and 20% by weight relative to the total weight of the composition or of the supplement.

The content of zinc gluconate in a cosmetic composition intended for oral administration or in a food supplement in accordance with the invention may be such that the daily dose of said zinc gluconate is between 0.01 and 300 mg/day, especially between 0.1 and 200 mg/day, and in particular between 1 and 100 mg/day.

According to yet another embodiment of the invention, the combination of active agents in accordance with the present invention comprises petroselinic acid and taurine, but is free of zinc or a salt thereof.

According to a preferred embodiment of the invention, the combination of active agents, the cosmetic composition intended for oral administration or the food supplement in accordance with the present invention comprises petroselinic acid, taurine and zinc or a salt thereof, preferably complexed with one or more (poly)hydroxy acids, in particular zinc gluconate.

According to one embodiment, a combination of active agents, a cosmetic composition for oral administration or a food supplement in accordance with the present invention comprises vitamin D3.

According to another preferred embodiment of the invention, the combination of active agents, the cosmetic composition intended for oral administration or the food supplement in accordance with the present invention comprises petroselinic acid, taurine, zinc or a salt thereof, in particular zinc gluconate, and vitamin D3.

According to one embodiment, a combination of active agents, a cosmetic composition for oral administration or a food supplement in accordance with the present invention comprises tocopheryl acetate.

According to one embodiment, a cosmetic composition for oral administration or a food supplement in accordance with the present invention may also comprise at least one vitamin chosen from vitamin B1, B5, B6, B8, B9, B12, C, D, and especially D3, PP, or tocopherol (vitamin E) and derivatives thereof, especially an ester such as tocopheryl acetate, succinate or palmitate, preferably tocopheryl acetate.

According to one embodiment, a cosmetic composition for oral administration or a food supplement in accordance with the present invention preferably comprises at least vitamin E or a derivative thereof and/or vitamin D, preferentially vitamin D3 and/or tocopheryl acetate.

According to a particular embodiment, a cosmetic composition for oral administration or a food supplement in accordance with the present invention comprises vitamin D3 and tocopheryl acetate.

Thus, according to a preferred embodiment of the invention, the present invention is directed toward a cosmetic composition intended for oral administration or a food supplement comprising petroselinic acid, taurine, zinc or a salt thereof, preferably complexed with one or more (poly)hydroxy acids, in particular zinc gluconate, vitamin D3 and tocopheryl acetate.

The compositions according to the invention may also comprise at least one carotenoid, especially a carotenoid chosen from β-carotene, lycopene, astaxanthin, zeaxanthin and lutein, flavonoids such as anthocyans, flavonols, flavanols (proanthocyanidins extracted from grape, catechins extracted from green tea), flavanones (hesperidin, diosmin), phenolic acids and derivatives (chlorogenic acid, from coffee extracts), diterpenes, stilbenes, chicory extracts, ginkgo biloba extracts, pimento extracts, soybean extracts, other sources of flavonoids with antioxidant properties, fatty acids, prebiotics, probiotics, resveratrol, amino acids, selenium and glutathione precursors.

The oral compositions or the food supplements in accordance with the invention may also comprise at least one probiotic, a prebiotic or a mixture of probiotics and a mixture of prebiotics. As probiotic microorganisms, mention may be made especially of Lactobacillus johnsonii or Lactobacillus paracasa.

The cosmetic compositions intended for oral administration or food supplements in accordance with the present invention may be in any oral-route galenical form normally used.

In the rest of the present text, when the limits x to y are discussed, the limits x and y are understood as being included.

Food Supplement

According to one embodiment, a cosmetic composition for oral administration or a food supplement in accordance with the invention comprises:

(i) petroselinic acid in a content of between 1% and 70% by weight, especially between 10% and 70% by weight and particularly between 20% and 70% by weight relative to the total weight of the combination of active agents;

(ii) taurine in a content of between 1% and 50% by weight, especially between 5% and 40% by weight and particularly between 10% and 30% by weight relative to the total weight of the combination of active agents; and/or

(iii) optionally at least one zinc (poly)hydroxy acid, preferably zinc gluconate, in a content of between 0.001% and 40% by weight, especially between 0.01% and 25% by weight and particularly between 0.1% and 20% by weight relative to the total weight of the combination of active agents;

(iv) optionally vitamin D3 in a content of between 0.0001% and 1.0% by weight, especially between 0.0001% and 0.5% by weight and particularly between 0.0001% and 0.1% by weight relative to the total weight of the combination of active agents; and/or

(v) optionally tocopheryl acetate in a content of between 0.01% and 10% by weight, especially between 0.1% and 10% by weight and particularly between 0.2% and 5% by weight relative to the total weight of the combination of active agents.

According to a particular embodiment, a cosmetic composition for oral administration or a food supplement in accordance with the invention comprises ingredients i) to v) below, taken together or individually:

(i) petroselinic acid in a content of between 1% and 70% by weight, especially between 10% and 70% by weight and particularly between 15% and 70% by weight relative to the total weight of the composition or of the supplement;

(ii) taurine in a content of between 1% and 40% by weight, especially between 5% and 40% by weight and particularly between 5% and 30% by weight relative to the total weight of the composition or of the supplement; and/or

(iii) optionally at least one zinc (poly)hydroxy acid, preferably zinc gluconate, in a content of between 0.001% and 30% by weight, especially between 0.01% and 25% by weight and particularly between 0.1% and 20% by weight relative to the total weight of the composition or of the supplement;

(iv) optionally vitamin D3 in a content of between 0.0001% and 1.0% by weight, especially between 0.0001% and 0.5% by weight and particularly between 0.0001% and 0.1% by weight relative to the total weight of the composition or of the supplement; and/or

(v) optionally tocopheryl acetate in a content of between 0.01% and 10% by weight, especially between 0.1% and 10% by weight and particularly between 0.2% and 5% by weight relative to the total weight of the composition or of the supplement.

According to a particular embodiment, the cosmetic composition for oral administration or the food supplement comprises all of the abovementioned ingredients (i) to (v).

Such a composition of food supplement type or an oral composition in accordance with the invention may in particular have the following contents:

| | % by weight relative to the total |
|---|---|
| Components | weight of the composition |
| Petroselinic acid | 54.9 (provided by the coriander seed oil) |
| Zinc gluconate | 6.3 (of which 13.6% of active material) |
| Taurine | 18.7 (of which 98.5% of active material) |
| Vitamin E | 1.0 (of which 67% of active material) |
| Vitamin D3 | 0.03 (of which 2.5% of active material) |

For ingestion, numerous embodiments of oral compositions and especially of food supplements are possible. Their formulation is performed via the usual processes for producing coated tablets, gel capsules, gels, emulsions, tablets, lozenges or soft capsules.

The compositions according to the invention, intended for oral administration, may especially comprise all or only a part of the daily dose.

In other words, one to three compositions may be administered per day.

Typically, the duration of this cosmetic treatment for oral administration may be greater than 4 weeks, especially from 4 to 24 weeks, with, where appropriate, one or more periods of stoppage.

The food supplement in accordance with the present invention may comprise one part of the active agents forming the combination according to the invention in a first composition, and the other part of these active agents in a second composition, as a kit or combination product for simultaneous use, separate use or sequential use over time.

This supplement may be formulated in such a way that the two compositions are in the same forms or in different forms, for example chosen from those mentioned above. Such a kit may in particular be provided in one and the same packaging.

Needless to say, a person skilled in the art will take care to select the optional additional additives and/or the amount thereof such that the advantageous properties of the combination according to the invention or of the composition comprising the combination according to the invention are not, or are not substantially, adversely affected by the envisaged addition.

The active agents according to the invention may be formulated with the usual excipients and components for oral compositions or supplements according to the invention, i.e. especially fatty and/or aqueous components, humectants, thickeners, preserving agents, texture agents and/or coating agents, antioxidants, flavorings and dyes that are common in the food supplement sector.

As cosmetic active agents other than the combination of active agents in accordance with the invention, a composition or a food supplement in accordance with the invention may contain an additional hydrophilic active agent chosen from sugars and sugar derivatives, water-soluble vitamins, plant extracts, for example from rosemary, pine bark or fruit such as orange, peptides and amino acids such as arginine, methionine, cysteine, citrulline and/or an additional lipophilic active agent chosen from retinol (vitamin A) and precursors thereof, especially beta-carotene, antioxidant carotenoids such as lycopene, zeaxanthin, astaxanthin and lutein, essential fatty acids, plant and animal oils, especially essential fish oils rich in omega 3, phospholipids such as lecithin, and mixtures thereof, oils from cucurbitacea plants such as marrow or sources of antioxidant enzymes such as melon, probiotics and prebiotics, vitamins and minerals, polyunsaturated fatty acids PUFA and monounsaturated fatty acids MUFA and omega 3 and omega 6 fatty acids.

Other characteristics and advantages of the invention will emerge more clearly from the examples that follow, which are given as non-limiting illustrations.

### EXAMPLE 1

Oral composition in soft capsule form.

| Ingredients | (mg/soft capsule) |
|---|---|
| Coriander seed oil | 300 |
| (65% petroselinic | |
| acid) | |
| Taurine | 76.10 |
| Zinc gluconate | 25.75 |
| Vitamin E | 4.10 |
| Vitamin D3 | 0.115 |
| Excipients | |
| Refined coconut oil | 112 |
| Yellow beeswax, | 22 |
| Cera flava | |
| Sunflower lecithin | 10 |
| Capsule | |
| Fish gelatin | 144.6 |
| Glycerol | 58.6 |
| Purified water | 6.8 |

### EXAMPLE 2

Oral composition in soft capsule form.

| Ingredients | (mg/soft capsule) |
|---|---|
| Coriander seed oil | 300 |
| (65% petroselinic | |
| acid) | |
| Taurine | 76.10 |
| Vitamin E | 4.10 |
| Excipients | |
| Refined coconut oil | 120 |
| Yellow beeswax, | 30 |
| Cera flava | |
| Sunflower lecithin | 12 |
| Capsule | |
| Fish gelatin | 160 |
| Glycerol | 58.6 |
| Purified water | 6.8 |

### EXAMPLE 3

Oral composition as a stick in emulsion form.

| Ingredients | (g/stick) |
|---|---|
| Coriander seed oil | 0.40 |
| (of which 65% of | |
| petroselinic acid) | |
| Taurine | 0.2 |
| Vitamin E | 0.0082 |
| Excipients | |
| Water | 1.722 |
| Sugar | 0.911 |
| Fructose | 0.911 |
| Microcrystalline | 0.032 |
| cellulose | |
| Sodium | 0.004 |
| carboxymethylcellulose | |
| Natural mixture of | 0.034 |
| tocopherols | |
| Sunflower oil | 1.015 |
| Natural lemon flavoring | 0.034 |
| Potassium sorbate | 0.013 |
| Citric acid | 0.013 |
| Propylene glycol | 0.01 |
| alginate | |

### EXAMPLE 4

Demonstration of the effect of a combination of petroselinic acid and taurine on the production of an endogenous anti-inflammatory resolvin derived from lipid metabolism: lipoxin A4 in an experimental model using human mononuclear cells in vitro.

Mononuclear blood cells are cultured under 5% CO₂ and at 37° C. in a serum-free medium for macrophages (SFM Macrophage; Invitrogen 12065074) for 24 hours. After this step, the medium is replaced with the same fresh test medium also containing the active agents at the various doses for 30 minutes in the presence of the various products to be evaluated (coriander oil 0.25 mg/ml, taurine (3.1 mg/ml). The inflammatory response was then triggered in the presence of phorbol myristate (0.05 μM) and calcium ionophore (1μM) and a lipid substrate mixture composed of docosahexaenoic acid (DHA−1 μg/mL) and eicosapentaenoic acid (EPA-1μg/mL).

The supernatants were then collected after 2 hours of stimulation and frozen at −80° C. before preparation for analysis by mass spectrometry.

Experimental triplicates (three wells) were prepared per experimental condition. Into each culture plate was placed a control corresponding to cells stimulated with the PMA/A23187 mixture and/or with addition of the equimolar mixture of fatty acids.

The frozen supernatants were concentrated by solid-phase extraction (SPE) and taken up in methanol before spectrometric analysis. The analytical method used consists in separating the various analytes by high-pressure liquid chromatography as a function of their retention time and in quantifying them by mass spectrometry.

The analyses were performed using an LC 1290 Infinity chain (Agilent Technologies) coupled to a 6460 Triple Quad LC/MS mass spectrometer (Agilent Technologies) equipped with an electrospray ionization source (Jet stream technology) operating in negative mode. The chromatographic separations were performed on a ZorBAX SB-C18 column.

The results were obtained in pg/mL of cell supernatant. These raw data were then transformed by calculation to obtain the percentage of activation (or of inhibition) of the plate relative to the control using the following calculation:

% modulation=100×(value obtained with the active agent—value of the control)/value of the control

These percentages of modulation are reported in the table below.

A combination of active agents in accordance with the invention comprising coriander oil, rich in petroselinic acid, and taurine, and also these same compounds individually, were tested in accordance with that indicated above.

The results obtained after these comparative tests are as follows:

| Compounds tested | Level of lipoxin A4 production |
|---|---|
| Coriander oil | +19% |
| (of which between 60% and 75% of | |
| petroselinic acid) | |
| 0.25 mg/ml | |
| Taurine | +38% |
| 3.1 mg/ml | |
| Taurine 3.1 mg/ml + coriander oil | +118% |
| (of which between 60% and 75% of | |
| petroselinic acid) 0.25 mg/ml | |

In this case also, it may be seen that the effect of a combination in accordance with the invention on the production of lipoxin A4 is very markedly greater than the sum of the effects of the compounds used individually.

Specifically, an increase in the production of lipoxin A4 of 118% relative to the basal level of production of this anti-inflammatory component could be seen when the lymphocyte cells tested were placed in contact with the combination of active agents.

It is thus indeed a synergistic effect of a combination of active agents in accordance with the invention that is observed and demonstrated here.

1-17. (canceled)

18. A cosmetic method for improving the quality of the head of hair, comprising at least a step consisting in an oral administration of a combination of active agents comprising petroselinic acid and taurine, wherein the improvement in the quality of the hair fiber comprises improving the tensile strength of the hair, and/or the resistance to styling and/or shaping of the hair, and/or preventing and/or combating limp and/or breakable and/or dull and/or split and/or embrittled and/or sensitized and/or dry hair, and/or improving the softness and/or vigor of the hair fibers.

19. The method as claimed in claim 18, for preventing and/or combating aging of the hair in the case of men over 30 years old or in the case of women.

20. The method as claimed in claim 18, for preventing and/or combating low-noise microinflammation of the hair follicles.

21. The method as claimed in claim 18, wherein petroselinic acid is used in the form of umbellifera plant oil or Geranium sanguineum oil.

22. The method as claimed in claim 18, wherein petroselinic acid is used in the form of coriander, chervil, carrot, celery, cumin, caraway, parsley or dill oil.

23. The method as claimed in claim 18, wherein petroselinic acid is used in the form of an oil of coriander seeds.

24. The method as claimed in claim 18, wherein the combination of active agents also comprises zinc gluconate.

25. The method as claimed in claim 18, wherein the combination of active agents also contains vitamin D3.

26. The method as claimed in claim 18, wherein the combination of active agents is used in the form of a food supplement.

27. The method as claimed in claim 26, in which the food supplement also comprises at least one vitamin chosen from vitamin B1, B5, B6, B8, B9, B12, C, D.

28. The method as claimed in claim 26, in which the food supplement also comprises at least vitamin D3 and/or tocopheryl acetate.

29. The method as claimed in claim 26, in which the food supplement also comprises at least one amino acid chosen from amino acids that are and/or are not constituents of proteins.

30. The method as claimed in claim 26, in which the food supplement also comprises at least one amino acid chosen from amino acids that are and/or are not constituents of proteins selected from arginine, cysteine and/or methionine.

31. The method as claimed in claim 26, in which the food supplement comprises a combination of petroselinic acid, taurine and zinc, or a salt thereof, and zinc gluconate.

32. The method as claimed in claim 26, in which the food supplement comprises petroselinic acid, taurine, zinc gluconate, vitamin D3 and tocopheryl acetate.

33. A food supplement containing a combination of petroselinic acid, taurine, zinc gluconate, the petroselinic acid being present in a content of between 20% and 70% by weight relative to the total weight of said combination of active agents.

34. The food supplement as claimed in claim 33, also comprising vitamin D3 and tocopheryl acetate.

35. The food supplement as claimed in claim 33, comprising:
(i) petroselinic acid in a content of between 1% and 70% by weight, relative to the total weight of the supplement;
(ii) taurine in a content of between 1% and 40% by weight, relative to the total weight of the supplement; and/or
(iii) at least one zinc (poly)hydroxy acid, in a content of between 0.001% and 30% by weight, relative to the total weight of the supplement; and/or
(iv) optionally vitamin D3 in a content of between 0.0001% and 1% by weight, relative to the total weight of the supplement; and/or
(v) optionally tocopheryl acetate in a content of between 0.01% and 10% by weight, relative to the total weight of the supplement.

36. A cosmetic process for improving the quality of the head of hair, comprising at least the oral administration to an individual of a combination of active agents as defined in claim 18 optionally in the form of a food supplement, wherein the improvement in the quality of the hair fiber comprises improving the tensile strength of the hair, and/or the resistance to styling and/or shaping of the hair and/or preventing and/or combating limp and/or breakable and/or dull and/or split and/or embrittled and/or sensitized and/or dry hair and/or improving the softness and/or vigor of the hair fibers.

37. The cosmetic process as claimed in claim 36, wherein it makes it possible to prevent and/or combat low-noise microinflammation of the hair follicles and/or of the scalp.

38. A food supplement comprising one part of the compounds forming the combination of active agents as defined in claim 18 in a first composition, and at least the other part of the compounds forming said combination of active agents in a second composition, as a kit or combination product for simultaneous use, separate use or sequential use over time.
